(19) United States
(12) Patent Application Publication
(10) Pub. No.: US 2012/0101481 A1
(43) Pub. Date: Apr. 26, 2012

(54) Device for Capsule Retrieval

(75) Inventor: Gordon C. Wilson, San Francisco, CA (US)

(73) Assignee: CAPSO VISION, INC., Saratoga, CA (US)

(21) Appl. No.: 12/987,740

(22) Filed: Jan. 10, 2011

Related U.S. Application Data
(60) Provisional application No. 61/405,167, filed on Oct. 20, 2010.

(51) Int. Cl. A61M 1/00

(52) U.S. Cl. 604/540

(57) ABSTRACT

A collection device for retrieving a medical capsule discharges from the anus of a patient is disclosed. Medical capsules have been widely used for monitoring and diagnostic purposes. The capsule has to be retrieved to obtain the on-board data or for reuse of the capsule. Accordingly, cost effective, easy-to-deploy, and comfortable-to-use collection devices are disclosed for retrieving the capsule. The capsule collection device for retrieving a capsule discharged from anus of a patient into a bowl comprises a capture device to capture the capsule and to pass feces discharged from the anus, and a wand to pick up the capsule. A collapsible capture device is also disclosure which comprises a flange to adapt to a rim of the bowl, a sifting piece to separate the capsule from the feces, and a collapsible side walls to couple the sifting piece to the capture opening of the flange. Furthermore, a wand having ejection capability for ejecting a picked capsule is disclosed.

### CROSS REFERENCE TO RELATED APPLICATIONS

The present invention claims priority to U.S. Provisional Patent Application No. 61/405,167, filed Oct. 20, 2010, entitled “Device for Capsule Retrieval.” The U.S. Provisional Patent Application is hereby incorporated by reference in its entirety.

### FIELD OF THE INVENTION

The present invention relates to medical capsule inside a human body. In particular, the present invention relates to retrieving the capsule upon its exiting from the human body.

### BACKGROUND

Medical capsules have been widely used for monitoring and diagnostic purposes. The capsule is swallowed by a patient and travels through the tract of the human body. When the capsule travels in the tract, it performs monitoring and/or diagnostic tasks such as measuring temperature and pH values, and capturing images. The measured or captured data may be transmitted to a device outside the body using wireless transmission or using the body as a transmission medium. Alternatively, the measured or captured data may be stored in a storage device within the capsule. The stored data can be recovered from the capsule after it exits from the body. In the latter case, there is a need for retrieving the capsule. Even for the capsule without on-board storage, there may still be a need for retrieving the capsule so that the capsule may be reused, or to prevent obstruction of the plumbing, or to comply with local laws governing disposal and recycling of devices containing batteries or other regulated components or materials.

When the capsule exits from the anus of a patient, it is often mixed with feces, where the feces include all excretions such as digested and partially-digested food, water, other liquids, and bile. It is desirable to design a cost effective, easy-to-deploy, and comfortable-to-use device that can be used to retrieve the capsule.

### BRIEF SUMMARY OF THE INVENTION

The present invention discloses a device for retrieving a medical capsule. The capsule collection device for retrieving a capsule discharged from anus of a patient into a bowl comprises a capture device to capture the capsule and to pass feces discharged from the anus, and a wand to pick up the capsule. The capture device comprises a flange to adapt to a rim of the bowl and a concave part joined to the flange around the capture opening, wherein at least one opening in the concave part to separate the capsule from the feces, and wherein the opening is smaller than the capsule. The flange contains a capture opening for collecting the feces. The concave part may comprise a sifting piece to separate the capsule from the feces and a side wall to couple the sifting piece to the capture opening. The wand comprises a shaft having a tip end and a handle end, and a magnet coupled to the tip end of the shaft to provide holding force for the capsule.

The present invention also discloses a collapsible capsule capture device for capturing a capsule and passing feces discharged from the anus of a patient. The collapsible capsule capture device comprises a flange to adapt to a rim of bowl and a concave part joined to the flange around the capture opening, wherein at least one opening in the concave part to separate the capsule from the feces, wherein the opening is smaller than the capsule, and wherein the concave part can be collapsed to conserve space. The flange contains a capture opening for collecting the feces. The concave part may comprise a sifting piece to separate the capsule from the feces and a side wall to couple the sifting piece to the capture opening. In order to conserve space for convenience of shipping and/or storage, the side walls can be collapsed.

The present invention further discloses a pick-up wand to pick up a capsule discharged from the anus of a patient. The wand comprises an inner shaft having a tip end and a rear end, a magnet coupled to the tip end of the inner shaft to provide holding force for the capsule, wherein the magnet is enabled to pick up the capsule at a first position and the capsule is disengaged from the magnet at a second position, an outer sleeve to allow the inner shaft to move between the first position and the second position in the outer sleeve by relative movement between the magnet and the outer sleeve, and a coupling means to couple the inner shaft with the outer sleeve to provide the first position and the second position. Furthermore, various coupling means embodying the present invention are disclosed.

### BRIEF DESCRIPTION OF THE DRAWINGS

FIG. 1A illustrates an off-the-shelf capture device that may be used to collect a capsule existing from a human body.

FIG. 1B illustrates an intended use of the off-the-shelf capture device of FIG. 1A adapted to a toilet bowl for collecting feces from a human body.

FIG. 2 illustrates a capture device converted from the off-the-shelf capture device of FIG. 1A and a wand for retrieving a capsule discharged from the anus of a patient.

FIG. 3 illustrates a collapsible capture device made of mesh fabric for retrieving a capsule discharged from the anus of a patient.

FIG. 4A illustrates another collapsible capture device for retrieving a capsule discharged from the anus of a patient.

FIG. 4B illustrates a bottom view of the collapsible capture device in FIG. 4A.

FIG. 4C illustrates the collapsible capture device in FIG. 4A in a partially collapsed position.

FIG. 4D illustrates the collapsible capture device in FIG. 4A in a fully collapsed position.

FIG. 4E illustrates a capture device having a porous wall and a non-porous base.

FIG. 4F illustrates a capture device having a continuous-piece concave part made of mesh fabric.

FIG. 4G illustrates a capture device having a continuous-piece concave part made of rigid or semi-rigid material.

FIG. 5A illustrates a foldable capture device for retrieving a capsule discharged from the anus of a patient, where the device is shown in a folded position.

FIG. 5B illustrates the foldable capture device in FIG. 5A in an unfolded position.

FIG. 5C illustrates a top view of the foldable capture device of FIG. 5B.

FIG. 6A illustrates another foldable capture device in an origami style for retrieving a capsule discharged from the anus of a patient, where the device is shown in an unfolded position.

FIG. 6B illustrates a top view of the foldable capture device in FIG. 6A.

FIG. 7A illustrates a scenario of using a wand to pick up a capsule collected by the capture device.

FIG. 7B illustrates a scenario of washing the capsule picked up by the magnet on the tip of the wand under running water.

FIG. 7C illustrates a scenario of discharging the capsule into a container by operating the ejector of the wand.

FIG. 8A illustrates an example of retractable wand comprising an outer sleeve and an inner shaft, where the wand is shown in a position to eject a capsule.

FIG. 8B illustrates the retractable wand of FIG. 8A in a position to pick up the capsule.

FIG. 9 illustrates a wand with the ejection capability comprising a shaft and an ejection sleeve, where the wand is shown in a position to pick up a capsule.

FIG. 10A illustrates another exemplary wand having the ejection capability comprising a shaft and an ejection sleeve, where the wand is shown in a position to eject a capsule.

FIG. 10B illustrates the wand of FIG. 10A in a position to pick up the capsule.

FIG. 11A illustrates an exemplary wand having the ejection capability comprising a shaft and an ejection sleeve with position locking, where the wand is shown in a position to eject a capsule.

FIG. 11B illustrates the wand of FIG. 11A in a position to pick up the capsule.

FIG. 12A illustrates a top view of another wand according to one embodiment of the present invention, where a pin is attached to the inner shaft to switch the position of the inner shaft.

FIG. 12B illustrates a cross section of the wand of FIG. 12A, where the inner shaft is shown in the retracted position.

FIG. 12C illustrates a cross section of the wand of FIG. 12A, where the inner shaft is shown in the enabled position.

FIG. 13A illustrates another example of retrieval kit embodying the present invention, where a wand with a capsule picked up by the magnet is inserted into a container for separating the capsule.

FIG. 13B illustrates a top view of the retrieval kit, where the structure on the top of the container consists of a wider region for inserting and a narrower region for disengaging the capsule.

FIG. 14 illustrates yet another example of retrieval kit embodying the present invention, where a wand with a capsule picked up by the magnet is ready for separating the capsule by partially closing the lid and retracting the wand.

FIG. 15 illustrates an example of alternative lid for the jar in FIG. 14 to facilitate capsule removal.

### DETAILED DESCRIPTION OF THE INVENTION

Medical capsules have been widely used for monitoring and diagnostic purposes. The capsule is administered by swallowing it by a patient and the capsule travels through the gastrointestinal (GI) tract of the human body. When the capsule travels in the tract, it performs its monitoring and diagnostic tasks such as measuring temperature and pH values, and capturing images. The measured or captured data may be transmitted to a device outside the body using wireless transmission or using the body as a transmission medium. Alternatively, the measured or captured data may be stored in an on-board storage device inside the capsule. The stored data can be recovered from the capsule after it exits from the human body. In the latter case, there is a need for retrieving the capsule. Nevertheless, even for capsules without on-board storage, there may still be benefits for retrieving the capsules so that the capsules may be recovered for reuse to save cost.

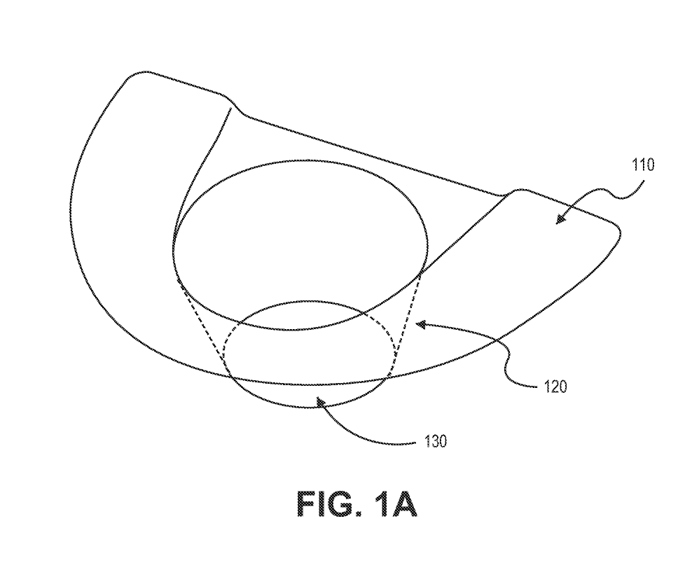

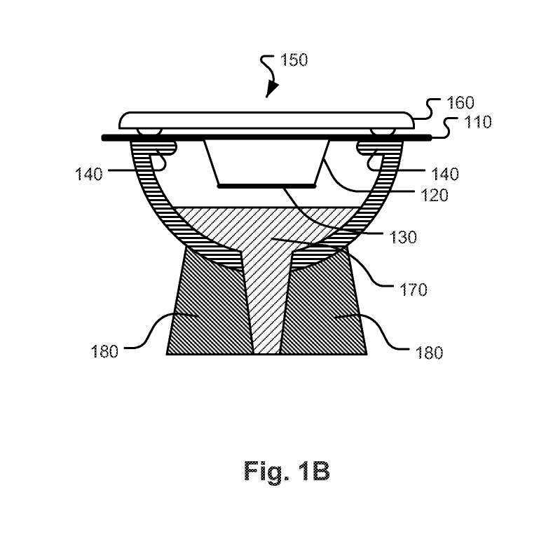

When the capsule exits from the anus of a patient, it is often mixed with feces, where the term, “feces” used in this disclosure, include all excretions such as digested and partially-digested food, water, other liquids, drugs, such as laxatives, or other substances that may be ingested by the patient, and bile. The capsule needs to be separated from the feces and preferably cleaned. The recovered capsule will be returned to a laboratory, preferably in a container, for recovering the data stored on-board or for reuse. Alternatively, the capsule may be disposed of in a manner preferable to flushing it down a toilet, for example, throwing it in the trash. The retrieval kit used to retrieve the capsule should be easy to deploy since it is mostly used by a patient without any special training in typical home environment. The cost of the retrieval kit is also a key design consideration since the retrieval kit is considered disposable. Therefore, the cost of the retrieval kit is directly added to the cost of each capsule administration. If the cost of the retrieval kit becomes too high, it reduces the incentive to reuse the capsule. Consequently, it is desirable to design a cost effective, easy-to-deploy, and comfortable-to-use retrieval device that can be used to retrieve the capsule. A straightforward method of retrieving the capsule may be using an off-the-shelf capture device such as the Kendall Specipan™ where the device is placed over the rim of a toilet bowl. FIG. 1A illustrates the structure of such capture device 100 where the flange 110 is used to adapt to a toilet bowl. The wall 120 and the bottom 130 form a cup to collect feces. FIG. 1B illustrates a cross-section view where the flange 110 of the capture device 100 is placed over the rim 140 of the toilet bowl 150. The toilet seat 160 is shown on the top of the flange 110, which implies that the toilet seat is put up when the capture device 100 is placed over the rim 140 and then the toilet seat 160 is put down. The water 170 in the toilet bowel and the base 180 of the toilet bowl are also shown in FIG. 1B. While FIG. 1B shows that the flange 110 is placed over the rim 140 and under the toilet seat 160, it is also possible to place the flange over the seat 160. In this disclosure, the flange 110 is placed “over the rim” may also refer to placing the flange 110 directly over the rim 140 or placing the flange 110 over the toilet seat 160 which is over the rim 140. After a capsule is administered into a patient, it is expected to use the capture device to catch the feces and possibly the capsule every time the patient has a bowl movement until the capsule is retrieved. The capsule has to be searched for through the feces using a hand with a glove on or using a tool with pick up capability. Furthermore, the feces may be diluted with liquid to make the search easier. On the other hand, too much liquid may cause it hard to spot the capsule under the un-cleared liquid. Therefore, this straightforward solution may serve the purpose of retrieving the capsule functionally. However, this method is not appealing to a patient.

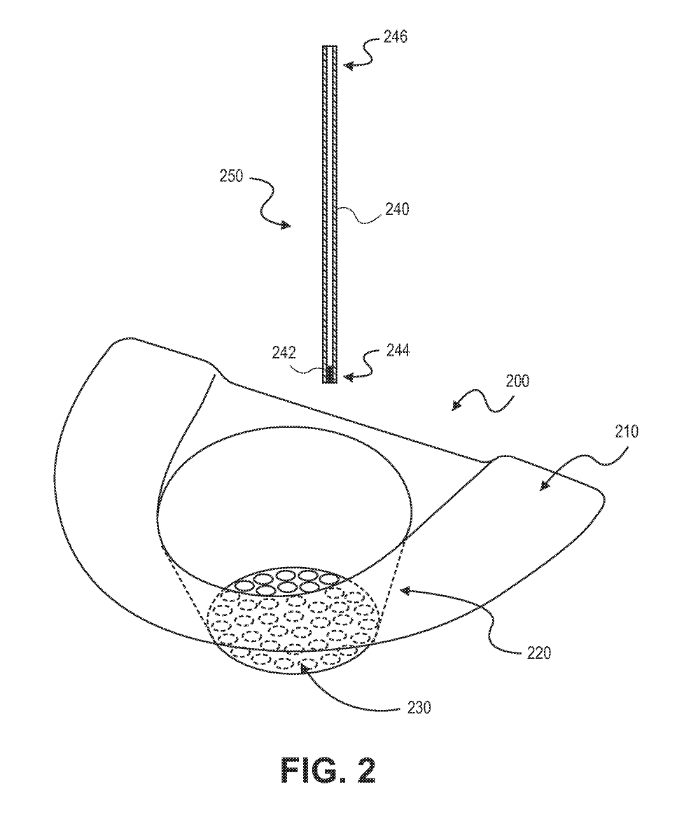

Accordingly, the present invention discloses a retrieval kit that can easily separate the capsule from the feces and can easily pick up the capsule. One exemplary embodiment according to the present invention is illustrated in FIG. 2. The retrieval kit comprises two parts: a capture device 200 and a wand 250. The capture device 200 comprises a flange 210 to be adapted to a toilet bowl by placing the flange 210 over the rim of the toilet bowl. The flange 210 may be made of a rigid part similar to the flange 110 of the capture device in FIG. 1A or a flexible material with a frame to support the flexible material. The bottom of the capture device is a sifting piece 230 which contains openings large enough to allow feces to pass through and small enough to prevent the capsule from passing through. The wall 220 is a structure to connect the flange and the sifting piece and keeping the capsule on the sifting piece 230. While a solid wall is shown in FIG. 2, the wall 220 can also be made of any flexible or solid material that is strong enough to connect the sifting piece 230 to the flange 210. The wall may also contain openings to allow liquid or feces to overflow or to reduce weight of the capture device. When the wall 220 contains openings, the opening should be small enough to prevent capsule from passing through.

While the capture device 200 in FIG. 2, adapted from an off-the-shelf capture device, comprises a distinct wall 220 and a distinct bottom piece 230, the cup-shape structure can be any concave part having openings to allow the feces to pass through. The cup-shaped structure will be able to collect feces discharged from the anus of a patient. The openings on the concave part will be able to allow the feces to pass through while retaining the capsule.

The wand 250 comprises a shaft 240 and a magnet 242, where the shaft 240 has a tip end 244 and a rear end 246 which serves as a handle. The magnet 242 is attached to the tip end 244. For these medical capsules containing metal parts, the magnet 242 will be able to pick up the capsule if the magnet 242 has enough holding force. A person can use the shaft 240 by holding the rear end 246 to pick up the capsule. After a patient makes a bowl movement over the capture device, water can be applied to help the feces to pass through the sifting piece 230 while retaining the capsule. Any container easily available at home can be used to collect water from a faucet and pour into the toilet bowl to allow the feces to pass through the sifting piece 230. Also, a squirt bottle may be used that can add water pressure and be directed to help the feces to pass through. An optional opening on the flange at a location between the wall 220 and the rim 140 of the toilet bowl can be made to allow toilet paper to pass.

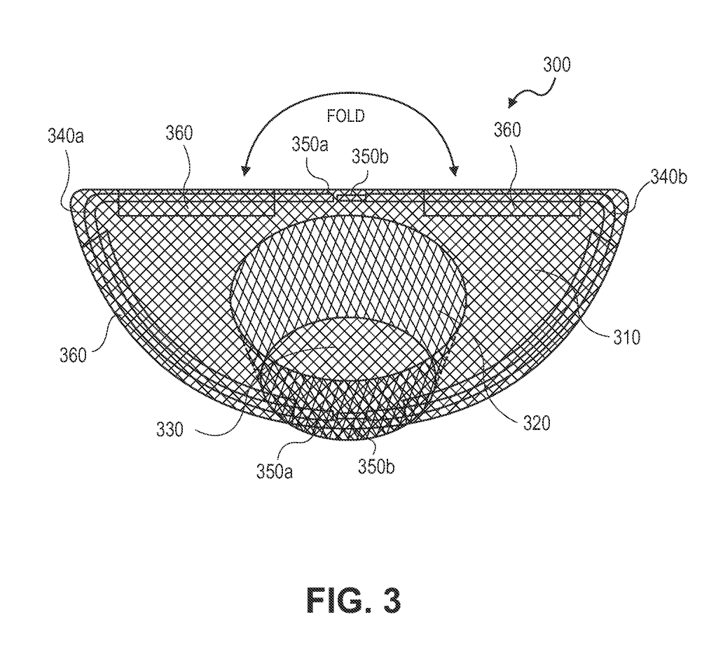

While a solid piece is shown in FIG. 2 for the flange 210, it may also be made of flexible materials such as mesh fabric or plastic. When a flexible material is used, a frame may be used to support the flange part, where a mesh fabric is used as an example of flexible material. The overall shape of the capture device is similar to that in FIG. 2. Since the material for the flange may be too flexible to support by itself, a frame structure can be used to extend the material for the flange so that it can support by itself. The frame can be made of material strong enough to extend the flexible flange material. For example, a cardboard having a shape of the flange border and having a center cutout may be used to keep the flange material extended. The border areas can be glued to the cardboard frame. Alternatively, the border areas of the flange may wrap around the cardboard frame to keep the flange extended. A tubular hoop may also be used as the frame to keep the flange extended. According to one embodiment of the present invention, a separable tubular loop may be used so that the capture device may be folded to conserve space during shipment or storage. FIG. 3 illustrates an example of a flexible capture device, where the capture device 300 comprises a flange 310 made of mesh fabric. A two-part separable tubular hoop, 340a and 340b, is used as an example where the two parts can be joined by joining the mating ends 350a-b. The mesh fabric can be formed as fasteners 360 to keep the frame in position. When the mating ends are separated apart, the capture device can be folded. When the mesh fabric is used for the flange, the same material may be used for the wall 320 and the sifting piece 330. Furthermore, the wall and the sifting piece can form a single-piece pocket jointly and there is no distinguishable boundary between the wall and the sifting piece. Furthermore more, the flange, the wall and the sifting piece can form a single piece jointly without any boundary among the flange, the wall and the sifting piece.

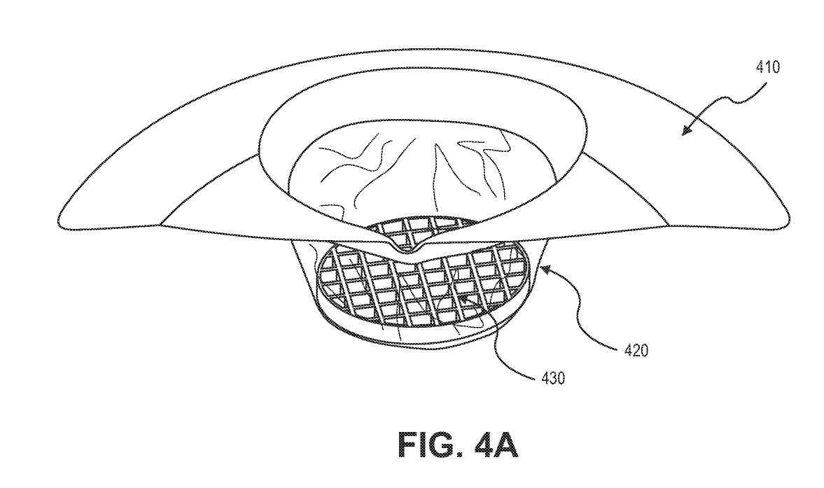

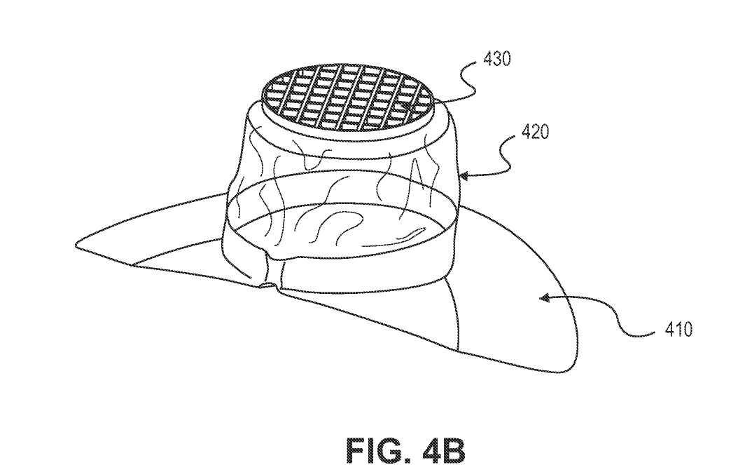

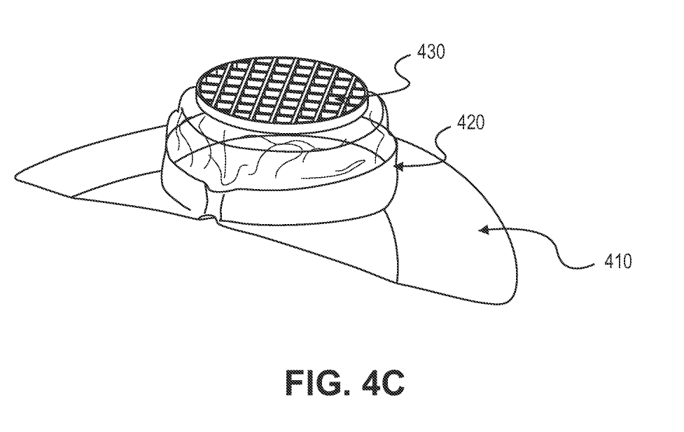

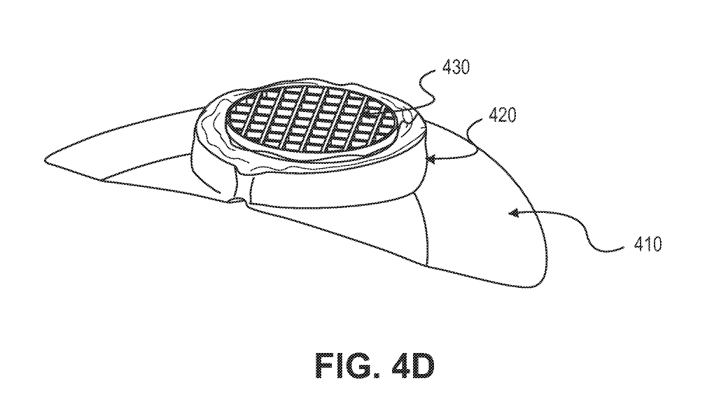

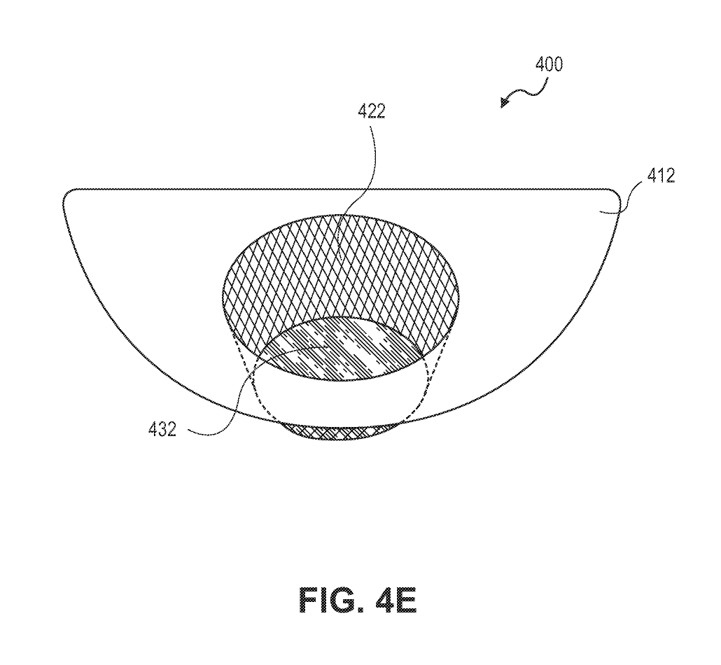

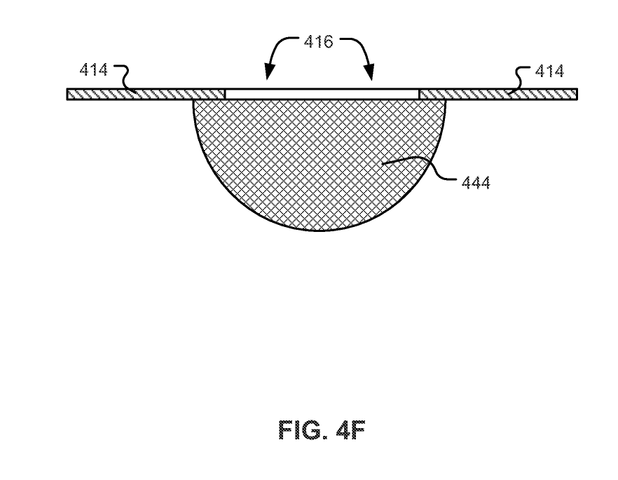

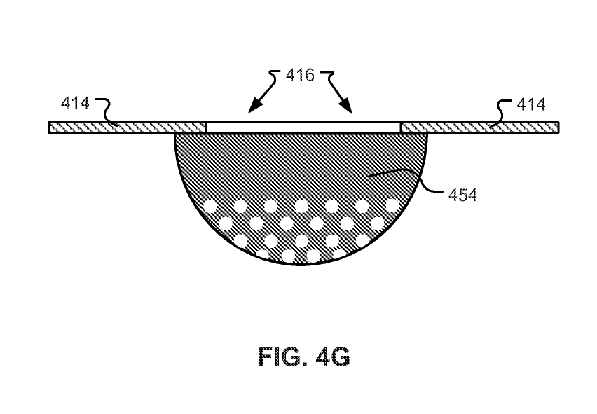

The capture device 200 in FIG. 2 may use a solid wall 220. The retrieval kit may have to be shipped to a patient and the packaging design for shipping convenience becomes a concern. With a solid wall, the capture device will have substantial vertical space and will require a sizable shipping box. It will be advantageous to use a flexible material for the wall so that the capture device becomes collapsible vertically. An embodiment according to the present invention is illustrated in FIG. 4A-FIG. 4D. FIG. 4A illustrates the collapsible capture device 400 in the upright position. The capture device 400 comprises a flange 410, a flexible wall 420 and a sifting piece 430. The collapsible capture device is shown in an upside down position in FIG. 4B. The material for the wall shown in FIG. 4A-FIG. 4D is made of flexible plastic sheet and the wall 420 can be collapsed by pushing the sifting piece 430 down when the collapsible capture device is in an upside down position or pushing the flange 410 down when the collapsible capture device is in an upright position. The wall 420 is shown in a partially collapsed position in FIG. 4C and fully collapsed in FIG. 4D. The wall may also be made of other flexible materials such as mesh fabric or soft cloth. When the flexible wall is made of a material substantially soft, the collapsible capture device may collapse by itself due to gravity. The sifting piece 430 shown in FIG. 4A-FIG. 4D is made of grate where the opening size is large enough for feces to pass through easily while small enough to retain the capsule. Other materials that contain openings of proper size and sturdy enough to sustain feces and liquid passing through it can also be used for sifting piece. For example, holes can be punched or drilled on a flat sheet of metal, plastic, cardboard, or other rigid material to make the material suitable for sifting piece. A material having the honeycomb structure can also be used as a material for the sifting piece as long as the opening size is large enough to pass the feces and small enough to retain the capsule. FIG. 4D illustrates a collapsible capture device in a fully collapsed position.

While the capture devices in FIG. 2 and FIGS. 4A-D have a sifting piece in the bottom to allow the feces to pass through, the bottom piece 432 can be made of non-porous material while the side wall 422 is made of porous material, particularly near the bottom portion of the side wall. The bottom piece 432 may also be called a base. FIG. 4E illustrates a capture device having a non-porous base and a porous side wall. The flange 412 may be made of rigid material such as the one in FIG. 2 or flexible material with a frame such as the one in FIG. 3. The side wall 422 shown in FIG. 4E is made of mesh fabric. However, the side wall 412 may also be made of rigid material, such as the one in FIG. 2. In this case, the holes are made on the side wall, particularly near the bottom of the side wall, instead of the bottom piece.

As mention previously that the concave part of the capture device does not have to contain a distinct side wall and a distinct base. The concave part may also be made from a continuous piece of material having holes. For example, the concave part can be made of continuous-piece mesh fabric is a pocket shape. FIG. 4F illustrates a side view of the capture device made of continuous-piece mesh fabric where the concave part 444 is attached to the flange 414 having a capture opening 416. Since the mesh fabric is flexible and can be collapsed, the capture device of FIG. 4E is also a collapsible capture device. While a flexible material can be used for the concave part, a rigid and semi-rigid continuous piece may also be used for the concave part. For example, a plastic bowel-shaped structure 454 can be used as the continuous piece for the concave part as shown in FIG. 4G.

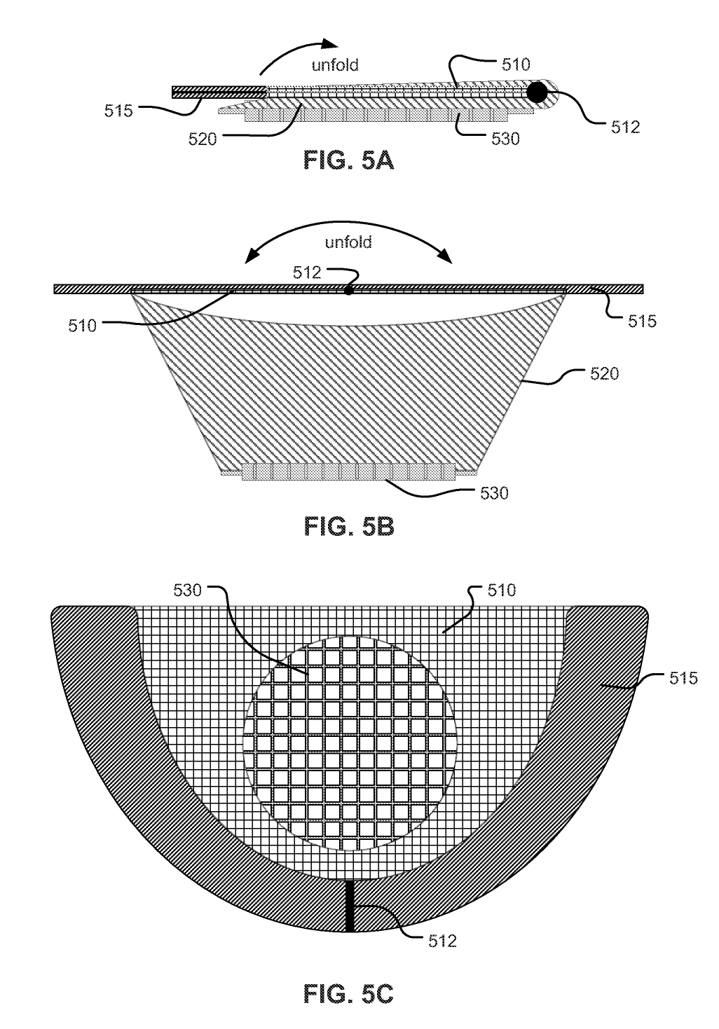

While the capture device conserves the vertical space, the flange 430 may still take up quite sizeable horizontal space. In order to further improve the compactness of the capture device for shipment, a capture device embodying the present invention comprises a foldable flange. A collapsible and foldable capture device 500 is shown in FIG. 5A in a collapsed and folded position. The flange 510 is made of flexible materials for easy folding. The flange contains a frame 515 having two sides joined by a locking hinge 512. The frame 515 is rigid to keep the flexible flange extended. FIG. 5B shows a side view and FIG. 5C shows a top view of the capture device 500, which comprises the flange 510, the locking hinge 512, the wall 520 and the sifting piece 530. While a locking hinge is used to make the flange foldable, other means commonly practiced in the field may also be used to make a foldable flange. For example, when rigid plastic is used as the material for the flange, a folding line can be formed by partially removing the material underneath the folding line so that the remaining material becomes flexible enough to fold.

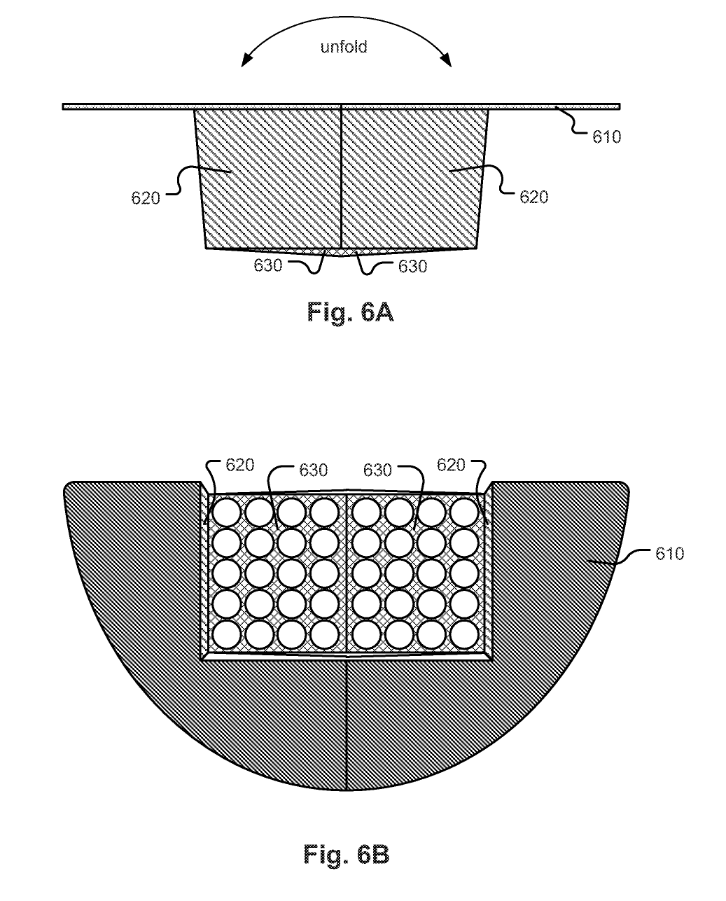

FIG. 6A and FIG. 6B illustrates yet another collapsible and foldable capture device. FIG. 6A illustrates a side view of the origami-style capture device 600, which comprises a flange 610, a wall 620 and a sifting piece 630. The capture device 600 comprising semi-rigid sections with creases and cuts for folding can be folded flat for easy to ship and to store. The material for the capture device 600 may be made of compostable plastic, heavy weight paper or waxed paper. Furthermore, the structure of the capture device may include snapping features for more rigidity.

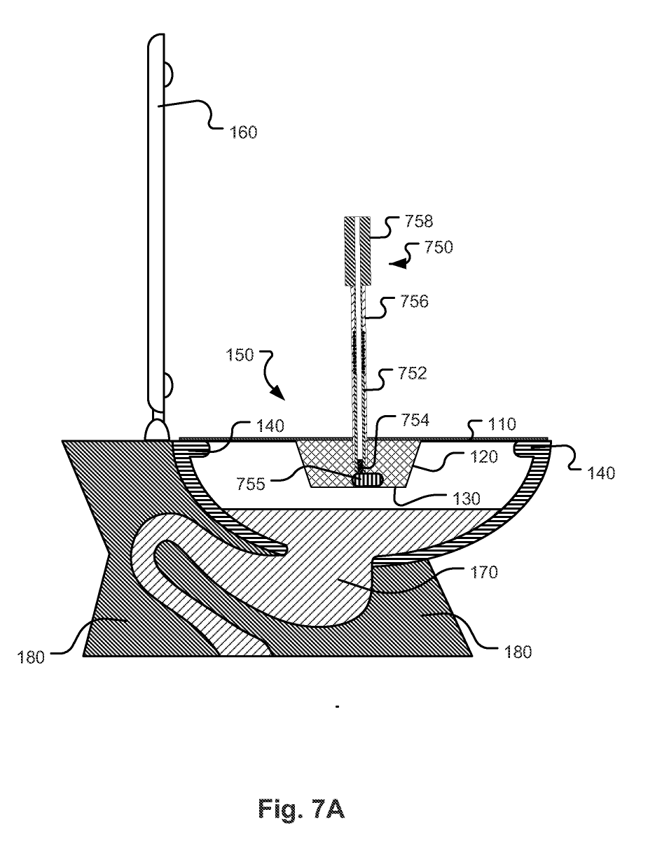

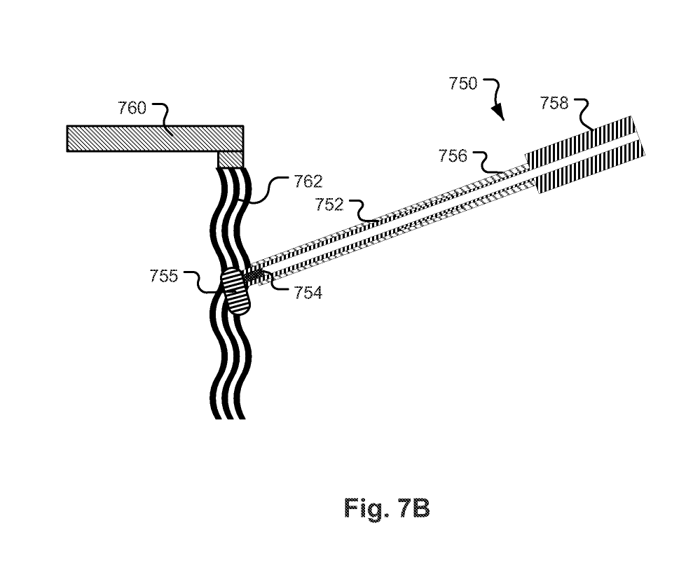

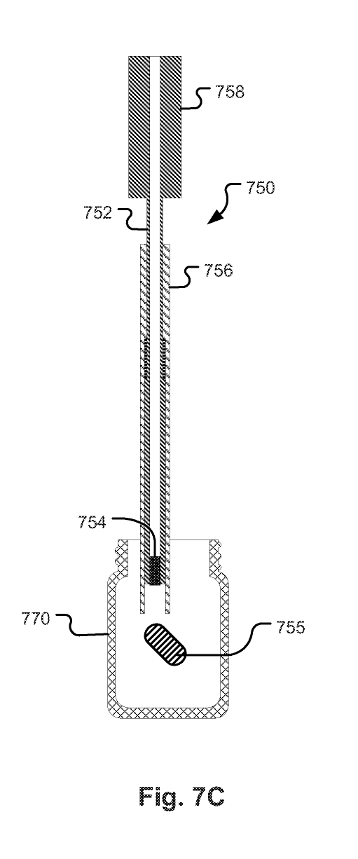

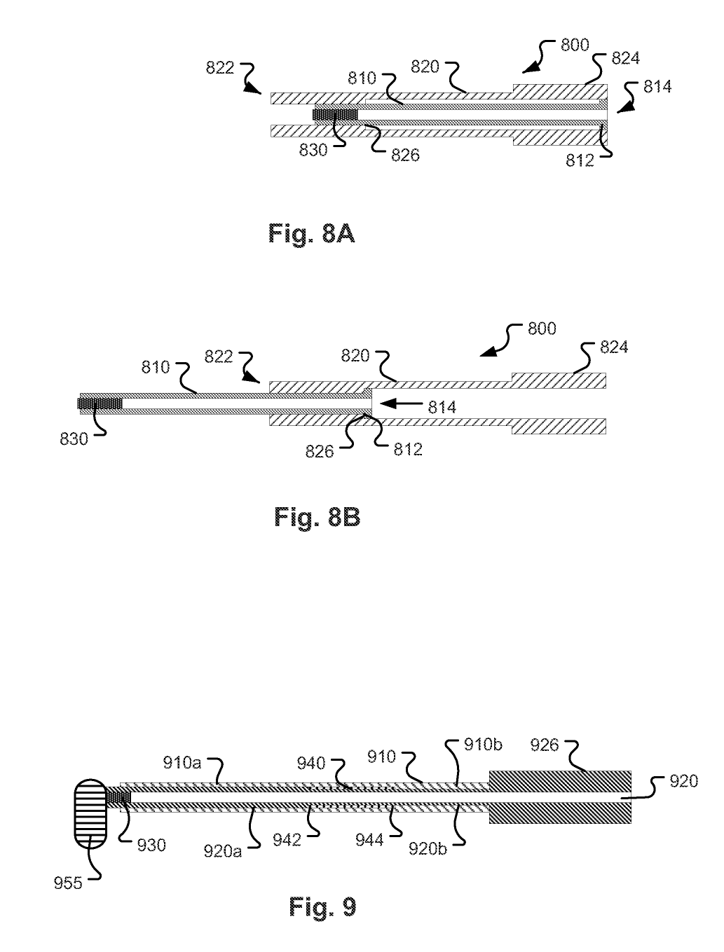

FIG. 7A-FIG. 7C illustrate a scenario that a capsule is retrieved after it exits from the anus of a patient. FIG. 7A illustrates a wand 750 is used to pick up a capsule 755 captured by the capture device 700, where the capture device 700 comprises a flange 710, a wall 720 and a sifting piece 730. As shown in FIG. 7A, the capture device is placed over a toilet bowl 150 by adapting the flange 710 over the rim 140 of the toilet bowl 150. The toilet seat 160 is shown in an upright position to avoid incidental water dripping on the toilet seat when picking up the capsule 755. The magnet on the tip of wand 750 is capable of picking up the capsule if a proper magnet with sufficient holding force is used. The wand 750 comprises an inner shaft 752, an outer sleeve 756, a magnet 752, and a handle 758. The capsule picked up with the wand 750 may be cleaned with running water 762 under a faucet 760 as shown in FIG. 7B or using a squirt water bottle. The cleaned capsule can be handled by hand to put it in a container such as a specimen bottle for returning to a laboratory for recovering on-board data or for reuse. Alternatively, the outer sleeve 756 of the wand 750 can be used as an ejector to eject the capsule into a specimen bottle 770 as shown in FIG. 7C. In the case that no ejection capability is needed, the wand still can be made retractable to save space for shipping and/or storage. FIG. 8 illustrates an example of retractable wand 800 comprising an inner shaft 810 and an outer sleeve 80. The outer sleeve 820 has a front end 822 and a handle 824. The inner side of the outer sleeve 820 contains a stopper 826 which can be engaged with the ring 812 at the rear end of the inner shaft 810 to stop the inner shaft 810 from moving forward beyond a point. One exemplary means for stopping the inner shaft 810 from moving beyond the extruded position in FIG. 8B is created by forming a stopper 826 with a smaller inner diameter at a section toward the front end and a larger inner diameter toward the rear end of the outer sleeve 820. The transition between the two different inner diameters forms the stopper 826. On the other hand, the ring 812 is formed by increasing the outer diameter of the inner shaft 810 near the rear end. Any other means for making a stopper can be incorporated to make the retractable wand. The inner shaft 810 comprises a magnet 830 on the tip of the front end where the magnet has enough holding force to pick up an intended capsule.

To handle the retrieved capsule by hand may not be comfortable for some patients and consequently a wand having the ejection capability is disclosed. FIG. 9 illustrates one embodiment according to the present invention. The wand 900 having the ejection capability comprises a shaft 920 and an ejection sleeve 910. A handle 926 is coupled to the rear end of the shaft 920 for ease grabbing by hand and also serving as a stopper for the ejection sleeve 910 in a fully retracted position. A spring coil 940 is installed between the inside of the ejection sleeve 910 and the outside of the shaft 920. The inside of the ejection sleeve 910 has two sections, where section 910a has a larger inner diameter and the section 910b has a smaller inner diameter. On the other hand, the outside of the inner shaft also has two sections, where the section 920 has a larger outer diameter and the section 910b has a smaller outer diameter to match with the inner diameters of the ejection sleeve 910. The spring coil 940 is coupled to the inner shaft 920 by interacting with the structure 942 having outer diameter transition. Also the spring coil 940 is coupled to the ejection sleeve 910 by interacting with the structure 944 having inner diameter transition. When the spring is in an un-forced condition, the magnet 930 on the front end of the inner shaft 920 will be extruded and is ready for picking up a capsule as shown in FIG. 9. To eject the capsule 955 picked up by the magnet 930, a user can pull the handle 926 backward while holding the ejection sleeve 910. Alternatively, a user can push the ejection sleeve 910 forward while holding the handle 926. The use of spring coil will increase manufacturing cost.

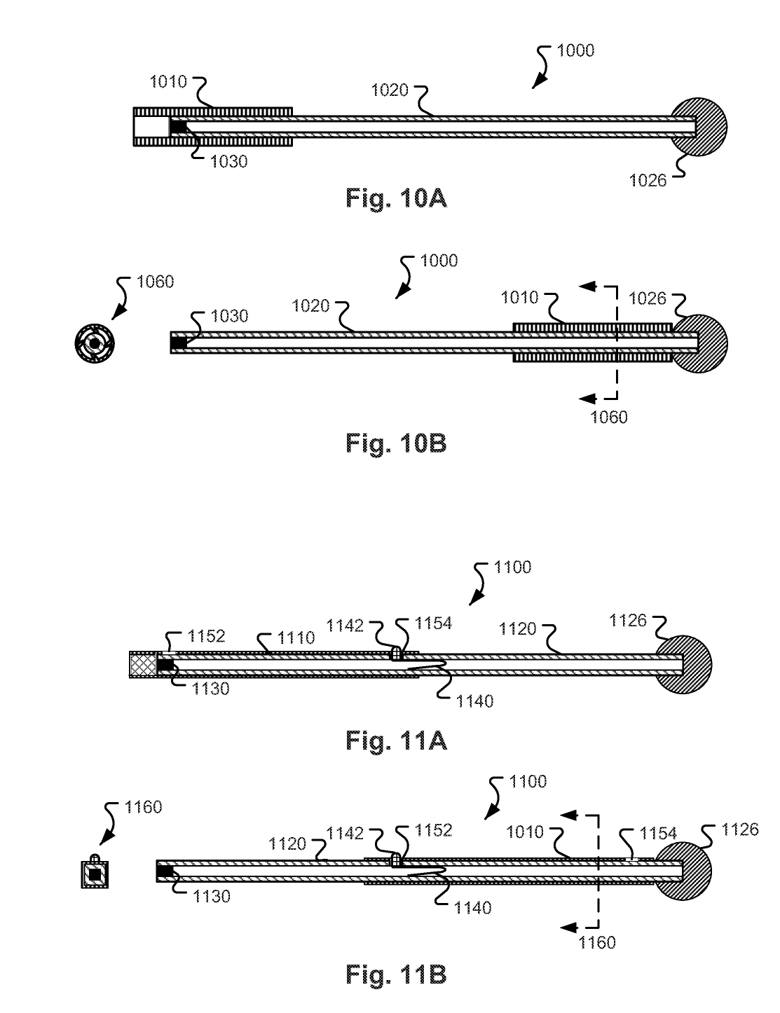

An alternative embodiment of the present invention is shown in FIG. 10A-FIG. 10B where the spring coil is not needed. The wand 1000 having the ejection capability comprises a shaft 1020 and an ejection sleeve 1010. A magnet 1030 is attached to the front end of the shaft 1020. A handle 1026 is coupled to the rear end of the shaft 1020 for ease grabbing by hand and also serving as a stopper for the ejection sleeve 1010. It is desirable to have the inner diameter of the ejection sleeve 1010 and the outer diameter of the shaft 1020 tightly controlled so that proper friction can be created between the ejection sleeve 1010 and the shaft 1020. To eject a capsule picked up by the magnet 1030, a user can pull the handle 1026 backward while holding the ejection sleeve 1010. Alternatively, a user can push the ejection sleeve 1010 forward while holding the handle 1026. Proper friction between the ejection sleeve 1010 and the shaft 1020 is important. Otherwise the ejection sleeve may easily fall off the shaft. Some structure in the inside of the ejection sleeve may be used to add friction between the ejection sleeve 1010 and the shaft 1020 without the need for precision manufacturing. The inner structure of the ejection sleeve can be seen in the cross section view 1060. Alternatively, some structure can be added to the outside of the shaft 1020 to add proper friction without the need for precision manufacturing. If neither the friction structure nor precision manufacturing is used, the wand in FIG. 10A can still be properly operated with caution by sliding the ejection sleeve 1010 toward the handle 1026 end as shown in FIG. 10B and grabbing both sleeve 1010 and the handle 1026 while picking up a capsule.

While the wand having ejection capability as shown in FIGS. 10A-B is easy to manufacture and is cost effective, the ejection sleeve 1010 may fall off the shaft 1020 due to insufficient friction during handling the capsule and causes the capsule to drop incidentally. Accordingly, another wand according to the present invention having the ejection capability is shown in FIGS. 11A-B where a snap button is used. The wand 1100 having the ejection capability comprises a shaft 1120 and an ejection sleeve 1110. A magnet 1130 is attached to the front end. A handle 1126 is coupled to the rear end of the shaft 1120 for easy grabbing by hand. The shaft 1120 is hollow at least for the section that a load spring 1140 with a snap button 1142 is installed. There are two holes 1152 and 1154 on the ejection sleeve 1110 to allow the snap button 1142 to rest. When the snap button 1142 rests in the hole 1152 or hole 1154, it will cause the wand locked in the position from extruding or retracting. The snap button 1142 can be pushed down to move the ejection sleeve between the two positions corresponding to hole 1152 and hole 1154. When the snap button rests in hole 1154, it corresponds to a retracted position as shown in FIG. 11A which can be the position when the wand is shipped or the capsule being ejected. When the snap button rests in hole 1152, it corresponds to an enabled position as shown in FIG. 11B which is the position that the wand is ready to pick up a capsule. When the shaft travels between the two positions, the shaft may be rotated and the snap button will not be aligned with the holes 1152 and 1154. To help the alignment of the snap button and the holes, a marker line in line with the snap button can be applied to the shaft. Alternatively, a non-round shaped shaft profile and matching inner profile of the ejection sleeve can be used to prevent the shaft from rotating inside the ejection sleeve. For example, the shaft may have a square or rectangular cross-section profile and the ejection sleeve will have the matching inner profile so that the shaft will not rotate when travelling inside the ejection sleeve. A cross section view 1160 of a wand having square shaft profile and matching sleeve is shown in FIG. 11B.

Hole 1154 may optionally be omitted. In this case, the shaft is released from the retracted position by pressing snap button 1142. The sleeve may then be freely moved down the shaft and over the tip to eject the capsule. At this point, the disposition of the sleeve is not critical. The sleeve may optionally be removed from the shaft or returned to a retracted position.

It may be desirable to unlock the sleeve from the shaft by manually pulling the two parts in opposite directions without the need to manually press the snap pin. If the snap pin extends through the shaft hole and the domed end of the pin enters the sleeve hole but does not penetrate so far that the cylindrical portion of the pin enters the sleeve hole, then the lateral force of the sleeve pushing against the pin will contact the domed portion and cause the pin to depress. In this embodiment, the pin prevents the sleeve from slipping off the shaft prematurely but does not firmly lock it in place. The operation is simplified for the patient as only a single ejection operation is needed (moving the sleeve over the shaft) instead of an unlocking and ejection operation.

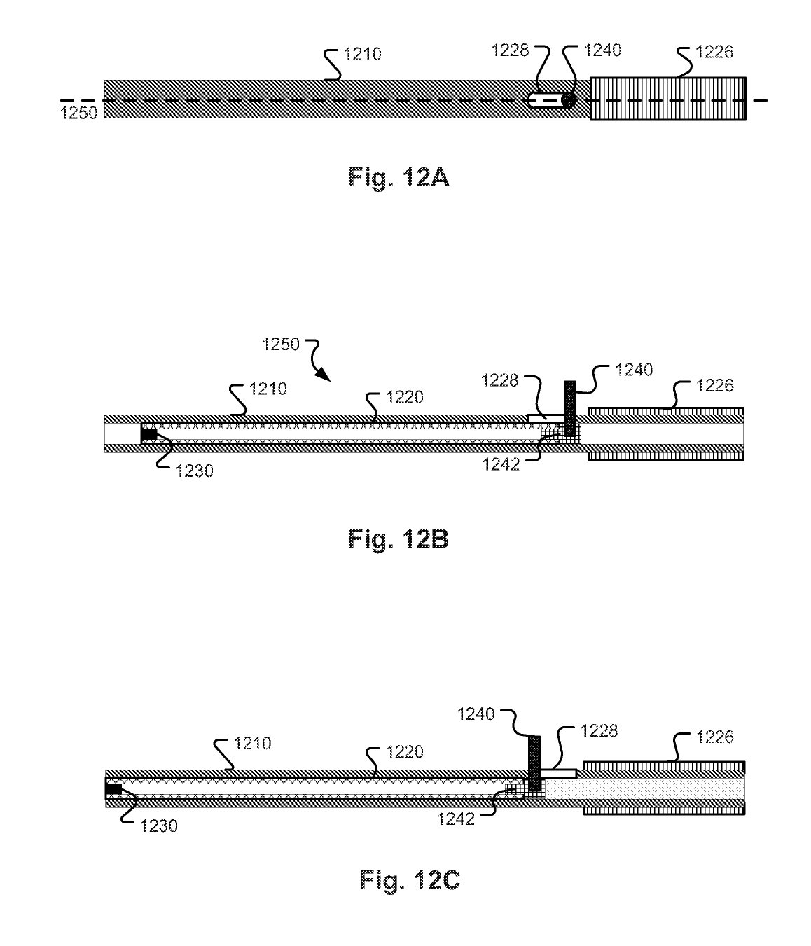

FIGS. 12A-C illustrate another embodiment according to the present invention. FIG. 12A shows a top view of the wand 1200, where the outer sleeve 1210 contains a slot 1228 and a handle 1226. A pin 1240 is attached to the inner shaft and extrudes from the slot 1228. FIG. 12B illustrates a cross section view 1250 of the wand 1200, where the inner shaft 1220 is shown in the retracted position with the pin 1240 positioned at the end of the slot 1228 toward the rear end of the outer sleeve 1210. FIG. 12C illustrates a cross section of the wand 1200, where the inner shaft is shown in the enabled position ready for picking up a capsule with the pin 1240 positioned at the end of the slot 1228 toward the front end of the outer sleeve 1210. The inner shaft 1220 comprises a magnet 1230 at the end toward the front end of the outer sleeve 1210. The pin 1240 is attached to the other end of the inner shaft 1220 through a joint 1242 so that when the pin 1240 is moved forward or backward, the inner shaft 1220 will be moved forward or backward accordingly.

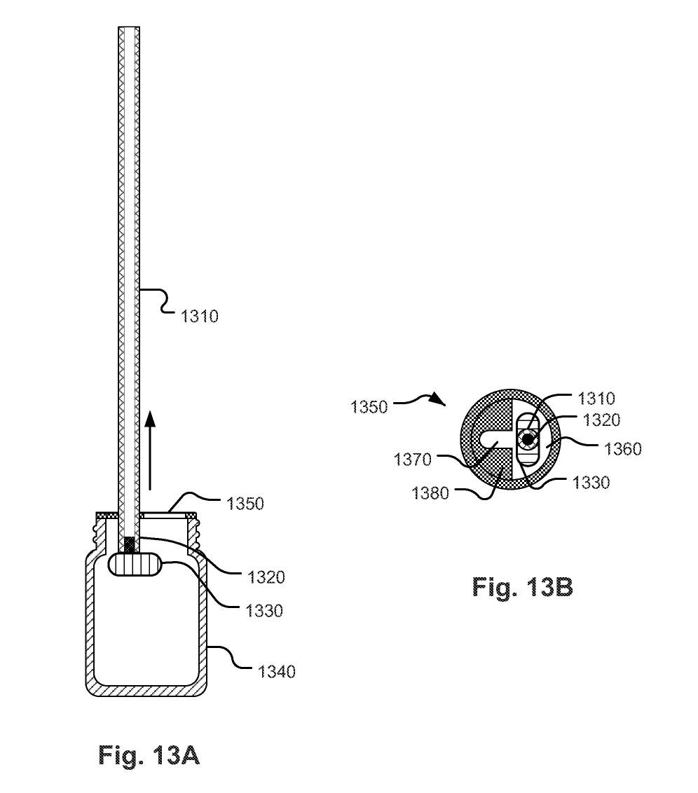

FIGS. 13A-B illustrate a kit embodying the present invention to strip off the capsule picked up by a wand into a container, where the capsule on the tip of the wand is moved into a container for stripping the capsule from the tip of the wand. While an ejector can be adopted in the wand as described above to separate the capsule from the wand, the retrieval kit in FIGS. 13A-B incorporates a structure placed on the top of a contains to strip the capsule from the wand. FIG. 13A shows a side view of the retrieval kit where the wand 1310 with the capsule 1330 picked up by a magnet 1320 is moved into the container to strip the capsule from the wand. FIG. 13B illustrates a top view of the retrieval kit, where the structure 1350 on the top of the container 1340 consists of a wider open region 1360 for the capsule to pass through and a narrower slot 1370 for stripping off the capsule. When the wand is located in the slot 1370, the capsule, at least in one orientation will be blocked by the non-open region of the structure 1350. The capsule on the tip of the wand can be moved into the container through the wider open region 1360 and then the wand is moved to the slot 1370 as shown in FIGS. 13A-B. Then, the wand can be drawn away as shown by the arrow in FIG. 13A and the capsule 1330 will be blocked by the structure 1380 and stripped off from the wand. While a distinct open region 1360 and a narrower slot 1370 are shown in FIG. 13B as an exemplary means to strip the capsule from the wand, any opening having one region wide enough for the capsule on the tip of the wand to go through at least in one capsule orientation and another region narrow enough to block the capsule in at least one capsule orientation from going through the structure. For example, a triangular shaped opening may be used so that the capsule on the tip of the wand can be moved through the opening along an edge of the triangle and be stripped off by moving the wand close to a vertex of the triangle and maintaining the capsule roughly in parallel with the edge opposite to the vertex. When the wand is pulled away from the contained, the capsule will be stripped off since the non-opening area linked to the two edges of the vertex will block the capsule from going through the opening.

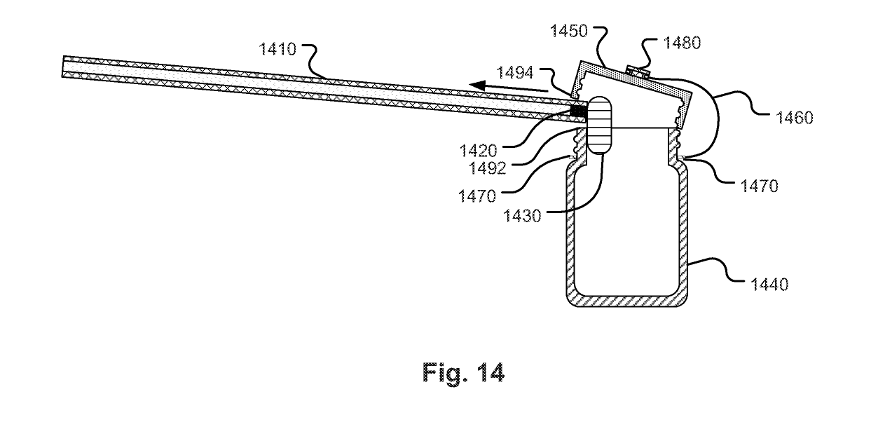

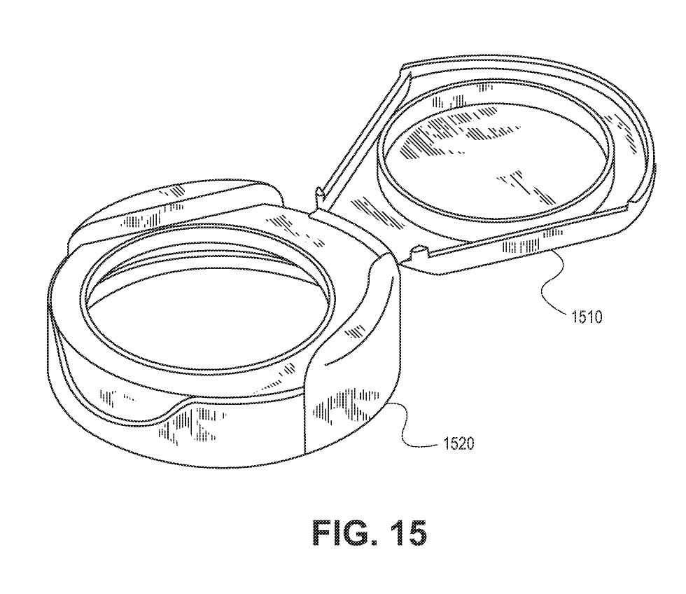

FIG. 14 illustrates yet another example of capsule stripping kit embodying the present invention, where a wand 1410 with a capsule 1430 picked up by the magnet 1420 is ready for stripping off the capsule by partially closing the lid 1450 and pulling the wand 1410 away from the container 1440. The lid 1450 may be snapped on or screwed on the container 1440. An optional strap 1460 may be used to attach the lid 1450 to the container 1440. A ring 1470 can be used to hold the strap 1460 to the container 1440. The strap 1460 may be attached to the spindle 1480 on the lid 1450 so that the lid 1450 can rotate freely which may be useful for the screw-on lid. To strip off the capsule 1430 from the magnet 1420 on the tip of wand 1410, the capsule 1430 on the wand 1410 is moved into the container 1440 with the lid 1450 off or widely ajar. While the capsule 1430 is inside the container 1440, the lid 1450 can be partially closed and the wand is tilted so that the space between the edge 1494 of the ajar lid 1450 and the respective edge 1492 of the container 1440 is small enough to prevent the capsule from getting out of the container. When the wand 1410 is pulled away from the contained 1440 as indicated by the arrow in FIG. 14, the capsule 1430 will be stripped off from the magnet 1420 and falls into the container 1440. FIG. 15 illustrates an example of alternative lid design that can be placed on the jar to facilitate capsule removal. The lid comprises a cover 1510 and a cap 1520 where the cap 1520 can be screwed on or snapped on the jar 1440. The cover 1510 can be snapped on the cap 1520 to close the opening. The capsule 1430 on the wand 1410 can be placed into the jar with cover 1510 open. The cover 1510 can then be half-way closed to prevent the capsule 1430 from getting out of the jar 1440. When the wand 1410 is pulled away from the jar, the capsule 1430 will be dropped into the jar.

In the disclosure, various embodiments and examples of the methods and structures mentioned above are described. It will be realized that this detailed description is illustrative only and is not intended to be in any way limiting. Other embodiments will readily suggest themselves to persons of ordinary skill in the art having the benefit of this disclosure.

1. A capsule collection device for retrieving a capsule discharged from the anus of a patient into a bowl, comprising:
a capture device to capture the capsule and to pass feces discharged from the anus; and
a wand to pick up the capsule;
wherein the capture device comprises:
a flange to adapt to a rim of the bowl, wherein the flange contains a capture opening for collecting the feces; and
a concave part joined to the flange around the capture opening, wherein there is at least one opening in the concave part to separate the capsule from the feces, and wherein the opening is smaller than the capsule; and
wherein the wand comprises:
a shaft having a tip end and a handle end; and
a magnet coupled to the tip end of the shaft to provide holding force for the capsule.

2. The capsule collection device of claim 1, wherein the flange includes a locking hinge to allow the flange to be folded.

3. The capsule collection device of claim 1, wherein the flange includes creases and cuts to allow the flange to be folded.

4. The capsule collection device of claim 1, wherein the concave part comprises a sifting piece to separate the capsule from the feces and a side wall to couple the sifting piece to the capture opening.

5. The capsule collection device of claim 4, wherein the sifting piece is made of a sifting material selected from a group consisting of rigid grate, mesh fabric, and flat sheet, wherein the sifting material has shifting openings smaller than capsule size.

6. The capsule collection device of claim 4, wherein the side wall is made of a wall material selected from a group consisting of rigid material and collapsible material.

7. The capsule collection device of claim 6, wherein the side wall has at least one hole, and wherein hole size is smaller than capsule size.

8. The capsule collection device of claim 1, wherein the concave part is made of porous material.

9. The capsule collection device of claim 8, wherein the porous material is mesh fabric.

10. The capsule collection device of claim 1, wherein the wand further comprises an outer sleeve to allow the shaft to move between a first position and a second position in the outer sleeve by relative movement between the magnet and the outer sleeve, wherein the magnet is enabled to pick up the capsule at the first position and the capsule is disengaged from the magnet at the second position.

11. A collapsible capsule capture device for capturing a capsule and passing feces discharged from the anus of a patient, comprising:
a flange to adapt to a rim of bowl, wherein the flange contains a capture opening for collecting the feces; and
a concave part joined to the flange around the capture opening, wherein there is at least one opening in the concave part to separate the capsule from the feces, wherein the opening is smaller than the capsule, and wherein the concave part can be collapsed to conserve space.

12. The collapsible capsule capture device of claim 11, wherein the concave part comprises a sifting piece to separate the capsule from the feces and a side wall to couple the sifting piece with the capture opening of the flange.

13. The collapsible capsule capture device of claim 12, wherein the sifting piece is made of a sifting material selected from a group consisting of rigid grate, mesh fabric, and flat sheet, wherein the sifting material has shifting openings smaller than capsule size.

14. The collapsible capsule capture device of claim 12, wherein the side wall is made of a wall material selected from a group consisting of flexible plastic, flexible cloth, and mesh fabric.

15. The collapsible capsule capture device of claim 11, wherein the flange includes a locking hinge to allow the flange to be folded.

16. A pick-up wand to pick up a capsule discharged from the anus of a patient, comprising:
an inner shaft having a tip end and a rear end;
a magnet coupled to the tip end of the inner shaft to provide holding force for the capsule, wherein the magnet is enabled to pick up the capsule at a first position and the capsule is disengaged from the magnet at a second position;
an outer sleeve to allow the inner shaft to move between the first position and the second position in the outer sleeve by relative movement between the magnet and the outer sleeve; and
a coupling means to couple the inner shaft with the outer sleeve to provide the first position and the second position.

17. The pick-up wand of claim 16, wherein a handle is coupled to the rear end of the inner shaft.

18. The pick-up wand of claim 16, wherein the coupling means comprises providing friction between the inner shaft and the outer sleeve by matching an outer diameter of the inner shaft with an inner diameter of the outer sleeve.

19. The pick-up wand of claim 16, wherein the coupling means comprises providing a coil spring between the inner shaft and the outer sleeve, wherein the spring coil is compressed to provide the first position and the spring coil is relaxed to provide the second position.

20. The pick-up wand of claim 16, wherein the coupling means comprises steps of:
providing an ejector over the inner shaft;
providing a first notch associated with the first position in the outer sleeve and a second notch associated with the first position in the inner shaft; and
providing a snap button passing through the second notch and entering the first notch to lock the inner shaft in the first position.

21. The pick-up wand of claim 16, wherein the coupling means comprises steps of:
providing an ejector over the inner shaft;
providing a slot in the outer sleeve; and
providing a pin attached to the inner shaft passing through the slot to move the inner shaft between the first position and the second position.

22. The pick-up wand of claim 21, wherein a handle is coupled to the outer sleeve.

23. A capsule stripping kit, comprising:
a pick-up wand to pick up the capsule discharged from an anus of a patient, wherein a magnet coupled to a tip end of the wand to provide holding force for the capsule;
a container having an opening to allow the capsule to be moved into the container; and
means for stripping off the capsule from the tip of the wand into the container.

24. The capsule stripping kit of claim 23, wherein the means for stripping off the capsule comprises a step of providing a structured piece at the opening of the container, wherein the structured piece comprises a first region wide enough for the capsule to pass through in at least one first orientation and a second region narrow enough for the capsule to be blocked by the structured piece from passing through in at least one second orientation.

25. The capsule stripping kit of claim 23, wherein the means for stripping off the capsule comprises providing a lid to cover the opening of the container, and configuring the lid in a position to partially close the opening of the container to prevent the capsule from passing through the partially closed opening.

26. The capsule stripping kit of claim 25, wherein the lid can be snapped on or screwed on the container over the opening of the container.

27. The capsule stripping kit of claim 25, wherein a strap is attached to a spindle on the lid and a ring on the container.
